(19) United States
(12) Patent Application Publication
(10) Pub. No.: US 2002/0133062 A1
(43) Pub. Date: Sep. 19, 2002

(54) Embedded measurement values in medical reports

(76) Inventors: Robert Stanley Arling, North Andover, MA (US); Brian Collamore, Rutland, MA (US); Sarah E. Jordan, Andover, MA (US); Sheila Filteau, Methuen, MA (US)

Correspondence Address: PHILIPS ELECTRONICS NORTH AMERICAN, 580 WHITE PLAINS ROAD, TARRYTOWN, NY, 10591, US

(21) Appl. No.: 09/808,002

(22) Filed: Mar. 15, 2001

(51) Int. Cl. A61B 5/00

(52) U.S. Cl. 600/300

(57) ABSTRACT

An electronically generated medical report, having an electronically selectable medical condition, and a field in the electronically selectable medical condition where, when the medical report is generated, a measurement corresponding to the field is retrieved and inserted into the field so that when the medical report is produced, the measurement replaces the field. The electronically generated medical report may also include multiple choice selections, arrangement selections and automated measurement selections.

### BACKGROUND OF THE INVENTION

1. Field of the Invention

The invention relates to a method and apparatus for modifying standard diagnoses and updating measurements in a diagnostic report of a patient's medical condition.

2. Description of the Related Art

When a patient undergoes a medical testing procedure, such as an ultrasound, the results of that procedure are recorded in a report. The report may be saved for future reference, or as an example of a medical condition. The creation of a report includes several standard procedures.

First, the patient is examined using a diagnostic device, such as an ultrasound machine. The raw results are then saved to a storage device. The storage device may be located on the diagnostic device or a network. A technician or physician then analyzes the raw results. The technician or physician can come to a conclusion that one or more medical conditions exist. The conclusions are then recorded and given to the patient's physician or the patient.

It is known to automate medical report generation through the use of a computer. In the various report automation devices, various medical conditions can be selected from a list of medical conditions. The medical conditions represent clinical diagnoses and other information relevant to the exam. The medical conditions can also represent the underlying pathology that might be encountered.

In addition, free text may be entered in a related section of the report to record diagnoses that are not part of the automatically prepared list. Free text entry can be recorded in a notes field that is included in an interpretation section of the report.

Another known report generation system feature enables the entry of measurement values into the report generation system such that various measurements pertaining to a patient's medical condition may be automatically inserted into a report. The measurements are usually included in a separate section of the report. The measurements may be typed in or may be derived by the report generation system from other user input, such as indicating points on a medical image between which distance or other metrics may be calculated.

In a medical report of the related art, it is possible to enter in a free text diagnosis that may contain a measurement value. The free text can then be manually modified. However, re-performing an automatically calculated measurement does not automatically change the free text portion of the report. Thus, measurements changed in a measurement section of the report may be inconsistent with the free text in the interpretation section of the report. In addition, the process of free text entry is error prone and may result in an incorrect measurement value being transcribed.

A second problem with the related art automated report is there is no way to modify a medical condition off a selected list such that a progressively more refined description may be used in diagnosing a medical condition. In the related art medical condition selection process, a user can scroll through a list of findings until a determined medical condition is found. At institutions having hundreds or even thousands of findings in a global list, several findings may vary only slightly from one another. The list of findings can thereby become very large and cumbersome to use, forcing users to search through an unnecessarily large and repetitive list in hopes of locating a specific item.

### SUMMARY OF THE INVENTION

A first exemplary embodiment is an electronically generated medical report, having an electronically selectable medical condition, and a field in the electronically selectable medical condition where, when the medical report is generated, a measurement corresponding to the field is retrieved and inserted into the field so that when the medical report is produced, the measurement replaces the field.

A second exemplary embodiment is a method of electronically generating a medical report, including electronically selecting a medical condition, and retrieving and inserting a measurement corresponding to a field in the electronically selectable medical condition when the medical report is generated wherein, when the medical report is produced, the measurement replaces the field.

Various other exemplary embodiments can have a list of a plurality of defined values associated with the measurement. Other exemplary embodiments can have the measurement replaced with a calculation value derived from one or more measurements. Other exemplary embodiments can have the measurement be at least one of a size, a qualifier, a link to a stored value, a time and a date. Other exemplary embodiments can have the report update the measurement at the time the report is generated and every time the report is viewed. Other exemplary embodiments can have the plurality of electronically selectable medical conditions be at least one of a pathology, an abnormality, an observation and a qualifier of a test result.

Another exemplary embodiment can have at least one additional medical condition that is displayed when the medical condition is selected. The original medical condition can be a category of a medical condition, and the second medical condition can be a specific medical condition.

### BRIEF DESCRIPTION OF THE DRAWINGS

The various objects and advantages of the invention will become apparent and more readily appreciated from the following description of the following exemplary embodiments, taken in conjunction with the accompanying drawings of which:

FIG. 1 is an exemplary embodiment of a report generation system according to the invention;

FIG. 2 is an exemplary method for producing a report according to the invention;

FIG. 3 is an exemplary block diagram of a screen showing measurement entry according to the invention;

FIG. 4 is a second exemplary block diagram showing diagnosis selection according to the invention;

FIG. 5 is an exemplary block diagram of the selection of a second diagnosis according to the invention;

FIG. 6 is a block diagram of how to rearrange the diagnosis according to the present invention; and

FIG. 7 is an exemplary report produced by the report generator.

### DETAILED DESCRIPTION

The following exemplary embodiments of the invention will be explained with reference to the accompanying drawings, wherein like reference numerals refer to like elements throughout.

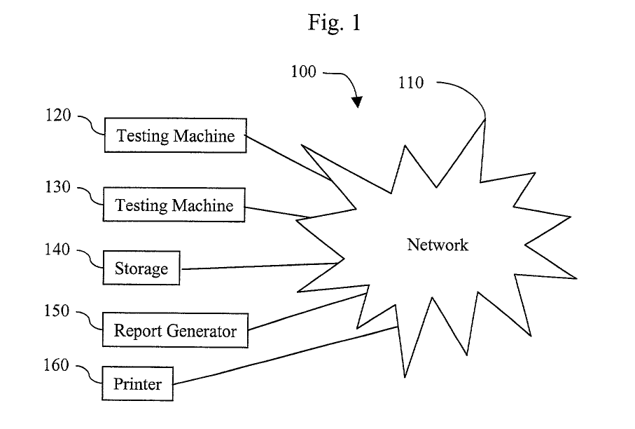

FIG. 1 illustrates the environment into which the invention can be used. In the system 100 there can be many testing machines 120, 130, a storage 140, a report generator 150 and a printer 160 connected to a network 110. The testing machines 120 and 130 represent many testing machines that may be connected to a network in a variety of methods as is well known in the art. The storage 140 represents both information stored on a testing machine, information separately stored on the network, and information stored on the report generator 150. The report generator 150 can be a personal computer, a specialized report generator, or any other machine known to automate report generation. The printer 160 can be a display screen, a laser printer, or any other device outputting or displaying a report as is well known in the art.

In exemplary method of using the system 100, a patient can first receive a test using the testing machine 120 or 130. The testing machine 120 or 130 can then transmit the results of the testing via the network 110 or other means such as removable media to the storage 140.

Users, for example a cardiologist, can then look at the information in the storage 140 and interpret the information as well as make diagnoses. The interpretations and diagnoses can either be stored on the report generator 150 or on the storage 140.

After the report has been finalized, the report can be sent to printer 160. Thus, the report generator 150 may generate multiple reports that may be the subject of later modifications and/or updates.

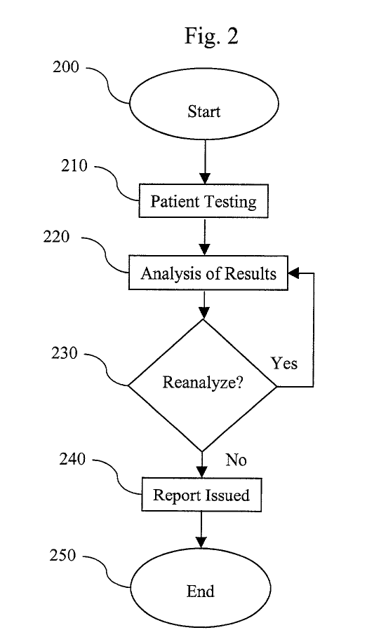

FIG. 2 illustrates the method of generating the report. The method can begin at start 200. The method then continues to patient testing 210.

In patient testing 210, physical parameters corresponding to the condition of a patient are recorded. These parameters may include an electrocardiogram, an EKG, or any other test as known in the art. The process then continues to analysis of results 220.

Analysis of results 220 includes both an automated and a manual entry of measurements from the results of the patient testing and the interpretation of the results of the patient testing. The method then continues to reanalyze 230.

If it is determined in reanalyze block 230 that another analysis of the results is required, the method jumps back to analysis of results 220. If it is determined that reanalysis is not necessary, the method continues to report issued 240.

In report issued 240, a report can be issued to either a screen or printed onto paper. The appropriate echo-cardiologist or physician can then sign the report. The method then continues to end 250 where it ends.

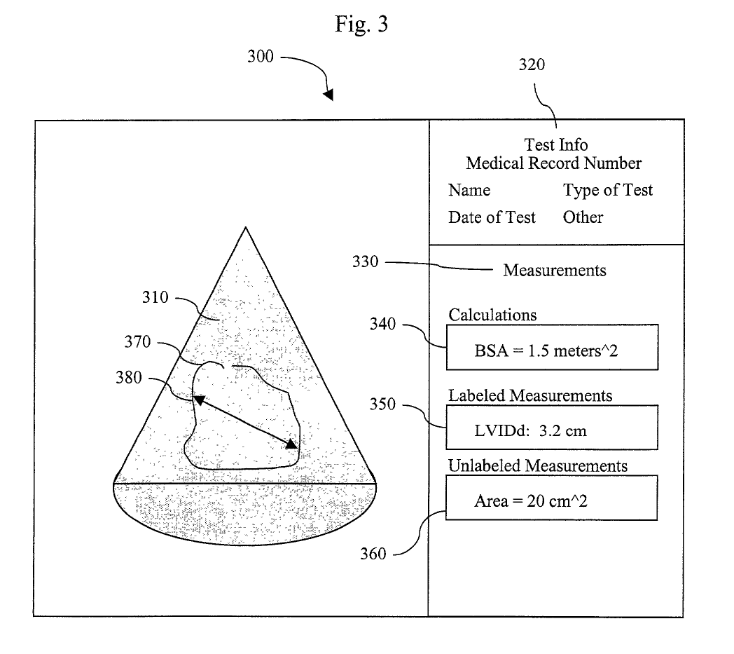

In FIG. 3 an exemplary screen showing an embodiment of the invention is displayed. A screen 300 can include a test information area 320, a measurement area 330, and testing information 310.

The test information area 320 can include information about the patient that generally does not change, such as a medical record number, a patient's name, the date of the test, the type of the test, or any other convenient information to be recorded about a patient.

The measurements area 330 can include sections for the entry of calculations derived from measurements 340, measurements identified with a label 350, and generic or unlabeled measurements 360. An example of a labeled measurement is LVIDd, which is the left ventricular internal dimension measured at end-diastole. An example of an unlabeled measurement is a generic length or area value.

A user may then enter information about various measurements within the various data entry blocks. The value of measurements and calculations may be automatically calculated by the report generation system based on various user inputs. These inputs may include typing in measurement values, drawing points on images between which distance or some other metric is calculated, electronically tracing regions in images from which areas are calculated, etc.

The testing information 310 may include objects representing various portions of a patient's anatomy such as heart shape 370. Additionally, the testing information 310 may include semi-automated data items such as a measurement 380. The measurement 380 may correspond to information entered into the measurement area 330. For example, the measurement 380 may represent 3.2 cm as entered into measurements identified with a label 350.

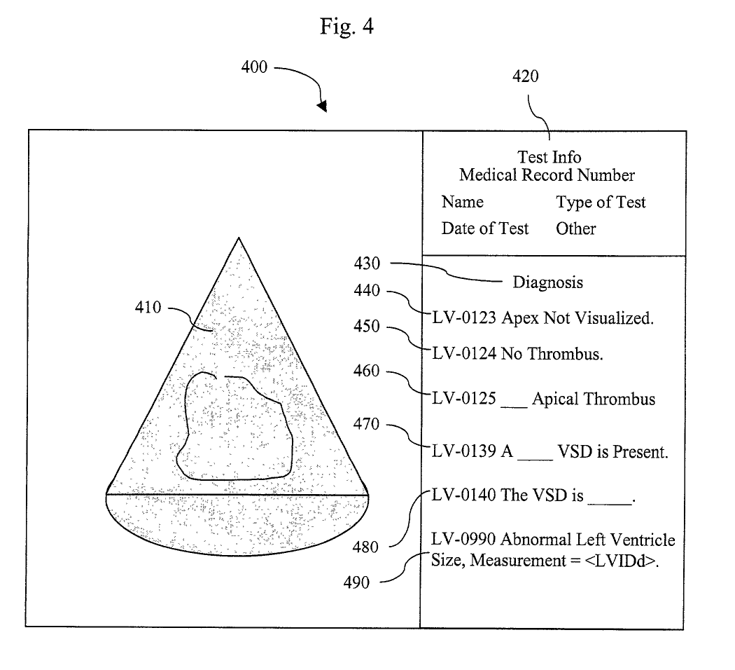

FIG. 4 illustrates another exemplary embodiment of the invention as shown in screen shot 400. Screen shot 400 may include test information 420, testing data 410, and a diagnosis 430.

The diagnosis 430 may include various analyses and/or diagnoses such as Apex Not Visualized 440, No Thrombus 450,_Apical Thrombus 460, a_VSD is present 470, the VSD is_480, and abnormal left ventricle size, measurement=<LVIDd>490.

Several of the exemplary diagnoses may be complete unto themselves such as No Thrombus 450. The report generator may then move directly onto the report generation or may wait for additional diagnoses to be entered.

Several other exemplary diagnoses may lead the user to additional diagnoses. For example, Apex not visualized 440 may direct the user to an additional screen where various other diagnoses and/or causes why the Apex is not visualized may be shown for a user to select.

Several other exemplary diagnoses may lead the user to a second screen with more information about a specific diagnosis. For example, A_VSD is present 470 may lead a user to an additional data entry screen where the type of VSD can be entered. The types of VSD may include muscular, membranous, or supracristal.

Other various exemplary diagnoses may include a diagnosis where a generic description of the diagnosis is entered. For example,_Apical Thrombus 460 may lead the user to a second screen where the user may pick among a small, a moderate size, or large thrombus. The qualifying value may be selected from a menu, a drop down box, a check box, or any other method used to select a single item from a plurality of choices.

Another exemplary embodiment may insert a measurement that may have previously been recorded in the measurement section 330 into the diagnosis. For example, abnormal left ventricle size, measurement=<LVIDd>490 may insert a measurement value of 3.2 cm into the=<LVIDd> section of the report.

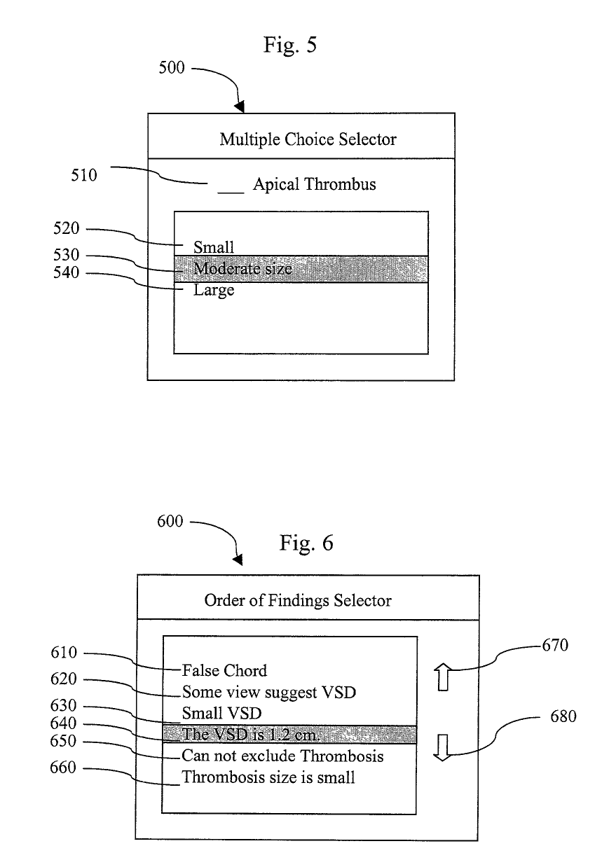

FIG. 5 illustrates a secondary Multiple Choice Selector of the current invention. A multiple-choice selector 500 may include a static section 510 and selectors small 520, moderate size 530, and large 540.

A user may thereby select one of the multiple choices from the multiple-choice selector 500. For example, a user may select large 540. Large 540 may then be replaced into the diagnoses, such that the report, when generated, reads Large Apical Thrombus.

FIG. 6 shows an Order of Findings Selector. The Order of Findings Selector 600 may include a diagnoses such as false chord 610, some view suggest VSD 620, small VSD 630, the VSD is 1.2 cm 640, Can Not exclude Thrombus 650 and Thrombus size is small 660. The Order of Findings Selector 600 may also include an up arrow 670 and a down arrow 680.

The user using the Order of Findings Selector 600 may rearrange the order of the findings for printing in the final report without modifying the earlier determined findings.

For example, the VSD 1.2 cm 640 maybe moved to the third position after Some view suggest VSD 620 by selecting the up arrow 670. Other means of reordering the findings, such as dragging and dropping the findings into new locations in the list, can also be employed.

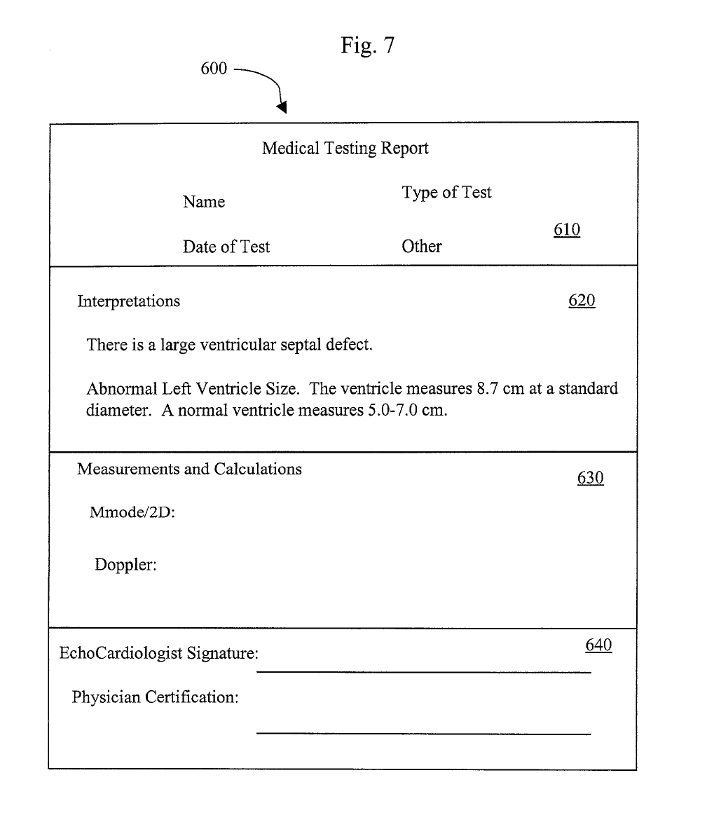

FIG. 7 illustrates an exemplary Medical Testing Report 600. Medical Testing Report 600 may include a static data section 610, an interpretation section 620, a measurements section 630 and a signature section 640.

The Static data section 610 may include information such as the name of the patient, the type of test, the date of the test or any other information as is well known in the art. The Interpretation section 620 may include information such as diagnosis or semi-automated data entry portions combined with interpretations. For example, the interpretations section may include the diagnoses of an abnormal left ventricle size. Further, the interpretation section may include the ventricle measures 8.7 cm at a standard diameter. Additional generic clauses may also be inserted that correspond to various diagnoses selected in the report generator. For example, the interpretation section 620 may include a comment that a normal ventricle measures 5.0-7.0 cm.

The measurements/calculations section 630 may include measurements and/or calculations either automatically or semi-automatically entered into the report generator. The measurements/calculations may be grouped by modality type. For example the measurements/calculations section 630 may include Mmode/2D, Doppler, or any other measurement or calculation as is known in the art.

The signature section 640 may include such items as the echo-cardiologist signature and the physician certification.

Thus, a first exemplary embodiment of the invention links the measurements section of the report with the interpretation section of the report. The exemplary embodiment can be a new type of finding which embeds a measurement value. The generic field name that corresponds to a measurement value, such as Left Atrial Dimension, can be inserted in the finding itself. Then, when the report is printed, the generic field name can be replaced by the actual value of the measurement, as recorded in the field.

In this exemplary embodiment, a user may select a finding that embeds the value of Left Atrial Dimension. The value displayed in the report will be linked to the previously recorded or computer measurement of <LA dimension>. Thus, if the user goes back and re-measures the Left Atrial Dimension, the report will update and remain consistent.

In addition, a second exemplary embodiment of the invention provides for the modification of list entries of a diagnosis of a medical condition. These modifications may be to link a selected medical condition to a more precise definition of the medical condition.

For example, a first exemplary embodiment of the invention allows for a user to select a medical condition known as Thrombus. The user can then modify the selected medical condition Thrombus by noting that the Thrombus is small, medium size, or large. This allows for medical conditions to be arranged in a tree-shaped scheme that allow for quick and efficient selection of a precise medical condition by a user.

Although exemplary embodiments of the invention have been described above and shown in the accompanying drawings, it will be appreciated by those skilled in the art that many changes and alterations to the embodiment are possible without departing from the principles and spirit of the invention.

What is claimed is:

1. An electronically generated medical report, comprising:
an electronically selectable medical condition; and
a field in the electronically selectable medical condition wherein, when the medical report is generated, a measurement corresponding to the field is retrieved and inserted into the field so that when the medical report is produced, the measurement replaces the field.

2. The apparatus of claim 1, further comprising a list of a plurality of defined values associated with the measurement.

3. The apparatus of claim 1, wherein the measurement is replaced with a calculation value derived from one or more measurements.

4. The apparatus of claim 1, wherein the measurement is at least one of a size, a qualifier, a link to a stored value, a time and a date.

5. The apparatus of claim 1, wherein the report updates the measurement at the time the report is generated and every time the report is viewed.

6. The apparatus of claim 1, further comprising at least one additional electronically selectable medical condition that is displayed when the electronically selectable medical condition is selected.

7. The apparatus of claim 1, wherein the electronically selectable medical condition is at least one of a pathology, an abnormality, an observation and a qualifier of a test result.

8. An apparatus comprising:
an electronic medical report which is electronically generated, the medical report including a medical condition having a corresponding field, wherein, when the medical report is generated, a stored, measured value for the medical condition is retrieved and inserted into the field so that the generated medical report is produced with the medical condition and the measured value.

9. The apparatus of claim 8, further comprising a list of a plurality of defined values associated with the measurement.

10. The apparatus of claim 8, wherein the measurement is replaced with a calculation value derived from one or more measurements.

11. The apparatus of claim 8, wherein the measurement is at least one of a size, a qualifier, a link to a stored value, a time and a date.

12. The apparatus of claim 8, wherein the report updates the measurement at the time the report is generated and every time the generated medical report is viewed.

13. The apparatus of claim 8, further comprising at least one additional medical condition that is displayed when the medical condition is selected.

14. The apparatus of claim 8, wherein electronically selectable medical condition is at least one of a pathology, an abnormality, an observation and a qualifier of a test result.

15. A method of electronically generating a medical report, comprising:
electronically selecting a medical condition; and
retrieving and inserting a measurement corresponding to a field in the electronically selectable medical condition when the medical report is generated wherein, when the medical report is produced, the measurement replaces the field.

16. The method of claim 15, further comprising listing a plurality of defined values associated with the measurement.

17. The method of claim 15, wherein the measurement is replaced with a calculation value derived from one or more measurements.

18. The method of claim 15, wherein the measurement is at least one of a size, a qualifier, a link to a stored value, a time and a date.

19. The method of claim 15, wherein the medical report updates the measurement at the time the report is generated and every time the report is viewed.

20. The method of claim 15, wherein the medical condition is at least one of a pathology, an abnormality, an observation and a qualifier of a test result.
